(19) United States
(12) Patent Application Publication
(10) Pub. No.: US 2012/0203137 A1
(43) Pub. Date: Aug. 9, 2012

(54) REDUCED-PAIN NEEDLE ASSEMBLY

(75) Inventor: Avishai NEUMAN, Valley Stream, NY (US)

(73) Assignee: AVISHAI NEUMAN, MD, PLLC, Valley Stream, NY (US)

(21) Appl. No.: 13/020,535

(22) Filed: Feb. 3, 2011

(51) Int. Cl. A61B 5/15

(52) U.S. Cl. 600/576

(57) ABSTRACT

Apparatus and methods are provided for use with a needle assembly that includes a needle that is for inserting into an insertion site of skin of a patient. A needle-coupling portion is coupled to a support element, the needle-coupling portion being configured to couple the support element to the needle assembly. A plurality of protruding members protrude from the support element, the protruding members being configured to engage the skin of the patient, and to be moveable with respect to the skin, while engaging the skin. Other embodiments are also described.

### FIELD OF EMBODIMENTS OF THE INVENTION

Some applications of the present invention generally relate to the insertion of a needle into a patient. Specifically, some applications of the present invention relate to apparatus and methods for reducing pain associated with piercing the skin with the needle during the insertion of the needle into the patient.

### BACKGROUND

Needles are used in medical practice for blood draws, immunization, administration of medication and saline delivery. Adults and children commonly find needle procedures to be frightening, painful, and/or otherwise distressing.

Psychotropic drugs, pain relief medication, and/or topical anesthetics may be administered to a patient in order to reduce fear, anxiety, pain and/or other unpleasant sensory and emotional experiences associated with actual or potential tissue damage, of a needle insertion. The administration of the aforementioned medications to the patient may be associated with risk to the patient. In addition to the aforementioned pharmacological interventions, patients may be subjected to cognitive-behavioral psychological interventions.

### SUMMARY OF EMBODIMENTS

For some applications of the present invention, a pain-reduction assembly is coupled to a needle assembly that includes a needle that is inserted into a patient's skin. Typically, the pain-reduction assembly is used to reduce pain associated with the insertion of the needle into the patient's skin, by bristles (and/or other protruding members) of the pain-reduction assembly being rubbed over the skin in the vicinity of the insertion site of the needle, prior to the needle being inserted into the insertion site. Typically, the rubbing of the bristles over the needle insertion site activates nerve receptors associated with the needle insertion site (or otherwise distracts the patient), which temporarily reduces the sensitivity of the needle insertion site to pain associated with the insertion of the needle.

There is therefore provided, in accordance with some applications of the present invention, apparatus for use with a needle assembly that includes a needle that is for inserting into an insertion site of skin of a patient, the apparatus including:

at least one support element;

a needle-coupling portion, coupled to the support element, the needle-coupling portion being configured to couple the support element to the needle assembly; and

a plurality of protruding members that protrude from the support element, the protruding members being configured to engage the skin of the patient, and to be moveable with respect to the skin, while engaging the skin.

For some applications, the protruding members define a serrated edge of the support element.

For some applications, the needle assembly includes a blood-receiving chamber that is configured to receive blood from the patient via the needle, and the needle-coupling portion is configured such that, when the support element is in a coupled state with respect to the needle assembly, a view of the chamber is provided through the needle-coupling portion.

For some applications, the protruding members are flexible and the protruding members are configured to be moveable with respect to the skin while engaging the skin, at least partially due to the flexibility of the protruding members.

For some applications, the support element is at least partially flexible and the protruding members are configured to be moveable with respect to the skin, while engaging the skin, at least partially due to the flexibility of the support element.

For some applications, the support element and the needle assembly form a single integrated unit, by the needle-coupling portion irreversibly coupling the support element to the needle assembly.

For some applications, the protruding members are configured to reduce pain associated with the insertion of the needle into the patient's skin, by being moved with respect to the skin.

For some applications, the protruding members are configured to tighten the patient's skin in the vicinity of the insertion site by engaging the skin in the vicinity.

For some applications, the protruding members are configured to at least partially obscure a view that the patient has of the needle by at least partially covering the needle.

For some applications, the protruding members are configured to reduce a likelihood of a healthcare professional suffering a needlestick injury from the needle, relative to the likelihood of a healthcare professional suffering a needlestick injury from a needle of a needle assembly that is not coupled to the support element, by the protruding members at least partially covering the needle.

For some applications, the apparatus further includes a substance disposed in a vicinity of the protruding members, the protruding members being configured to apply the substance to the skin, the substance being selected from the group consisting of an anesthetic, an analgesic, and an antiseptic.

For some applications, the substance is disposed on at least some of the protruding members.

For some applications, at least some of the protruding members are shaped to define hollow spaces therein, the substance being disposed inside the hollow spaces.

For some applications, the apparatus further includes an absorbent material configured to be disposed in the vicinity of the protruding members, the substance being configured to be absorbed in the absorbent material.

For some applications, the needle-coupling portion is configured to reversibly couple the support element to the needle assembly.

For some applications, the needle-coupling portion includes a snap-on mechanism configured to couple the support element to the needle assembly.

For some applications, the needle-coupling portion includes an adhesive configured to couple the support element to the needle assembly.

For some applications, the needle assembly includes a plurality of needle-assembly types, and the needle-coupling portion is configured to couple the support element to any one of the plurality of needle-assembly types.

For some applications, the support element is configured such that when the support element is in a coupled state with respect to the needle assembly, a view of the needle is provided through the support element.

For some applications, the support element is shaped to define a space, and the support element is configured such that, when the support element is in a coupled state with respect to the needle, access to the needle is provided via the space.

For some applications, the support element includes a U-shaped support element that defines the space via which the access to the needle is provided.

For some applications, the support element includes two arms, the arms being configured to be disposed on respective sides of the needle, when the support element is in a coupled state with respect to the needle.

For some applications, the arms are flexible and are configured to tighten the patient's skin in the vicinity of the insertion site by engaging the patient's skin.

For some applications, the protruding members include twenty or more particles of a coarse material that is disposed on the support element.

For some applications, the twenty or more particles include twenty or more sand particles of sandpaper that is disposed on the support element.

For some applications, the protruding members include twenty or more bristles that protrude from the support element.

For some applications, at least one of the bristles includes a fiber-optic bristle, and the apparatus further includes at least one light source configured to facilitate visualization of the patient's skin by directing light via the fiber-optic cable.

For some applications, the apparatus further includes:

at least one fiber-optic cable configured to be placed inside at least one of the bristles; and

at least one light source configured to facilitate visualization of the patient's skin by directing light via the fiber-optic cable.

There is further provided, in accordance with some applications of the present invention, apparatus for use with a needle assembly that includes a needle that is for inserting into an insertion site of skin of a patient, the apparatus including:

at least two flexible elongate elements; and

a needle-coupling portion configured to couple the elongate elements to the needle assembly, the needle-coupling portion being configured to couple the elongate elements to the needle assembly, such that the elongate elements are disposed on respective sides of the needle,

the elongate elements being configured to stretch the skin in the vicinity of the insertion site, by flexing laterally away from one another, while engaging the skin in the vicinity.

There is additionally provided, in accordance with some applications of the present invention, a method for use with a needle assembly that includes a needle that is for inserting into an insertion site of skin of a patient, the method including:

placing on the patient's skin, in a vicinity of the insertion site, a plurality of protruding members that protrude from at least one support element, the support element being coupled to the needle assembly; and

subsequently, inserting the needle into the skin, while simultaneously rubbing the skin in the vicinity of the insertion site with the protruding members, by simultaneously advancing distally with respect to the skin in the vicinity, the needle and the support element.

There is further provided, in accordance with some applications of the present invention, a method for use with a needle assembly that includes a needle that is for inserting into an insertion site of skin of a patient, the method including:

placing on the patient's skin, in a vicinity of the insertion site, at least two flexible elongate elements, the elongate elements being coupled to the needle assembly such that the elongate elements are disposed on respective sides of the needle;

stretching the skin in the vicinity by laterally flexing the elongate elements away from one another; and

while the skin is stretched, inserting the needle into the insertion site.

The present invention will be more fully understood from the following detailed description of embodiments thereof, taken together with the drawings, in which:

### BRIEF DESCRIPTION OF THE DRAWINGS

FIGS. 1A-B are schematic illustrations of a needle and catheter being inserted into a patient's skin, the needle having been coupled to a pain-reduction assembly, in accordance with some applications of the present invention;

FIGS. 2A-C are schematic illustrations of bottom views of the pain-reduction assembly, respectively, before a needle assembly is coupled to the pain-reduction assembly, during the coupling of a needle assembly to the pain-reduction assembly, and subsequent to the coupling of the needle assembly to the pain-reduction assembly, in accordance with some applications of the present invention;

FIGS. 3A-B are schematic illustrations of top views of the pain-reduction assembly, respectively, before a needle assembly is coupled to the pain-reduction assembly, and subsequent to the coupling of the needle assembly to the pain-reduction assembly, in accordance with some applications of the present invention;

FIGS. 4A-B are schematic illustrations of side views of the pain-reduction assembly, respectively, before a needle assembly is coupled to the pain-reduction assembly, and subsequent to the coupling of the needle assembly to the pain-reduction assembly, in accordance with some applications of the present invention;

FIGS. 5A-B are schematic illustrations of a pain-reduction assembly having flexible support arms, in accordance with some applications of the present invention; and

FIGS. 6A-B are schematic illustrations of a pain-reduction assembly having support arms that are joined to one another at distal ends thereof, respectively, before a needle assembly is coupled to the pain-reduction assembly, and subsequent to the coupling of the needle assembly to the pain-reduction assembly, in accordance with some applications of the present invention.

### DETAILED DESCRIPTION OF EMBODIMENTS

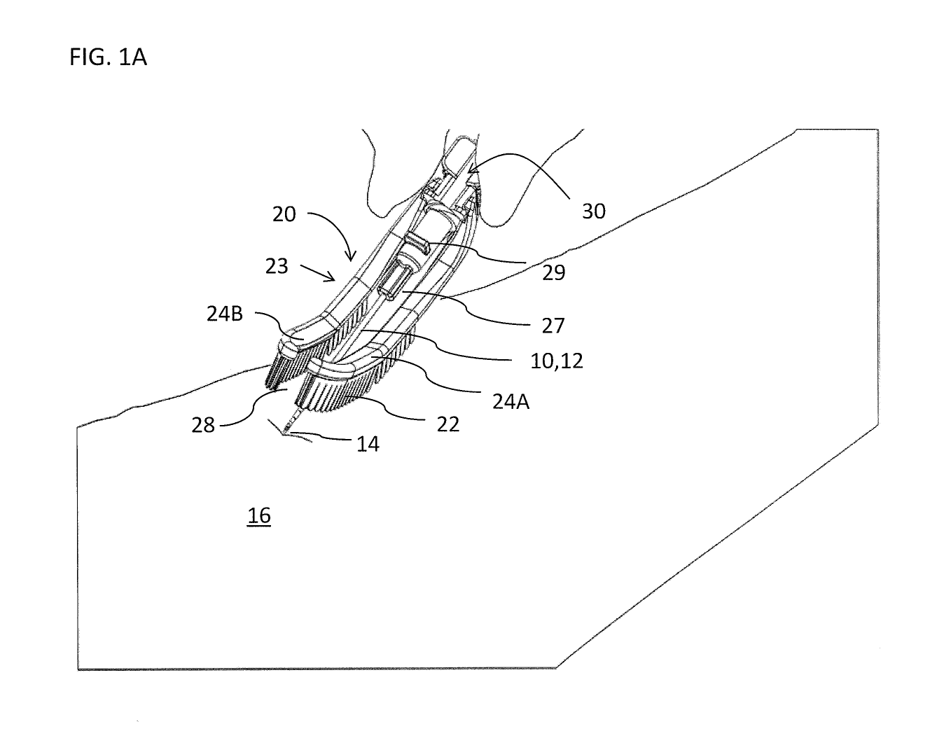

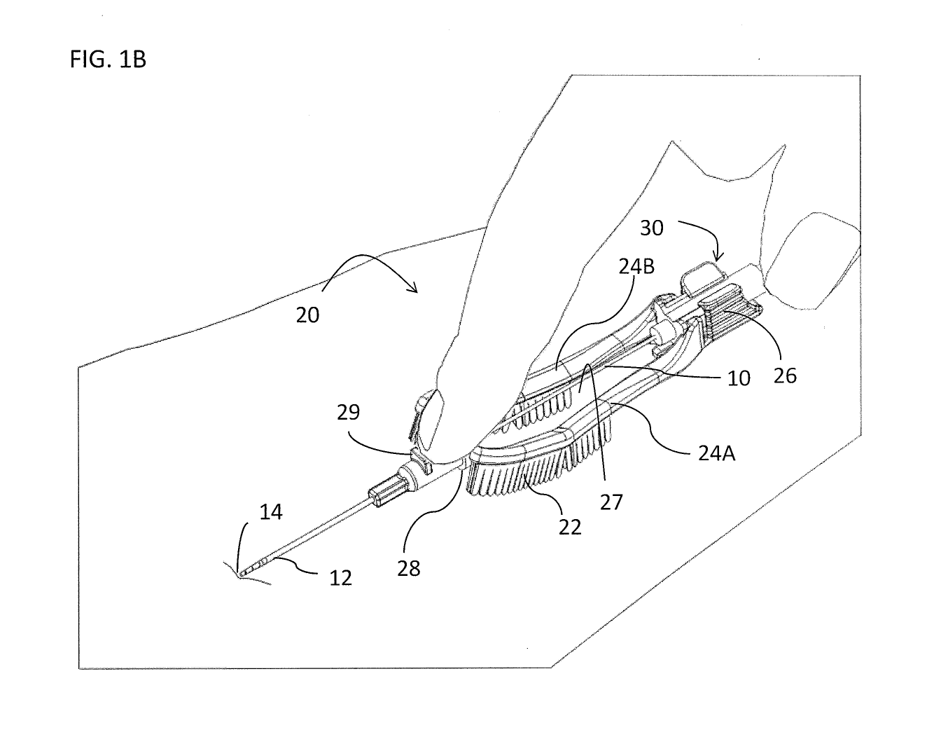

FIGS. 1A-B are schematic illustrations of a needle and a catheter 12 of a needle assembly 30 being inserted into an insertion site 14 of a patient's skin 16, the needle assembly having been coupled to a pain-reduction assembly 20, in accordance with some applications of the present invention. Pain-reduction assembly 20 typically includes a plurality of (e.g., at least twenty) protruding members for rubbing the patient's skin, e.g., fibrous or metal bristles 22, shown in FIG. 1A. The bristles are supported by a support element 23, e.g., support arms 24A and 24B, the support arms being configured to be disposed on respective sides of needle 10, subsequent to the coupling of the pain-reduction assembly to the needle. A needle-coupling portion 26 (shown in FIG. 1B) is coupled to the support element, and is configured to couple the pain-reduction assembly to the needle, e.g., by needle-coupling portion 26 being coupled to a portion of a needle-assembly 30, the needle assembly including needle 10.

Typically, pain-reduction assembly 20 is used to reduce pain associated with the insertion of needle 10 into the patient's skin, by bristles 10 being rubbed over the skin in the vicinity of insertion site 14 prior to the needle being inserted into the insertion site. Typically, the rubbing of the bristles over the needle insertion site activates nerve receptors associated with the needle insertion site (or otherwise distracts the patient), which temporarily reduces the sensitivity of the needle insertion site to pain associated with the insertion of the needle. In a typical technique, in accordance with some applications of the present invention, the bristles are placed on the patient's skin. Subsequently, the needle assembly and the pain-reduction assembly are simultaneously advanced distally with respect to the patient's skin, such that (a) the skin is rubbed by the bristles, and simultaneously (b) the needle is inserted into insertion site 14.

Alternatively or additionally, bristles 22 are used to obscure the view that the patient has of the needle, thereby reducing patient anxiety associated with the insertion of the needle into the skin. Further alternatively or additionally, the bristles reduce needlestick injuries, by at least partially covering the tip of the needle before the needle is placed over the insertion site. Typically, before the needle is placed over the insertion site, support element 23 and bristles 22 extend beyond the distal end of the needle, thereby at least partially covering the distal end of the needle.

Pain-reduction assembly 20 is typically configured to be coupled (e.g., reversibly coupled) to off-the-shelf needle assemblies, via needle-coupling portion 26. For example, assembly 20 may be couplable to any venipuncture needle assembly, e.g., a blood-draw needle, an injection needle, a Vacutainer® needle, a Vacuette® needle, a butterfly needle, and/or a syringe needle. Alternatively or additionally, assembly 20 may be coupled to an intravenous therapy device (i.e., an “IV device”), a fingerstick needle, a chemoport access needle, a dialysis access needle, and/or any other medical needles that are known in the art. For some applications, pain-reduction assembly 20 is formed and packaged for sale by the manufacturer as a single integrated device together with needle assembly 30. For example, needle-coupling portion 26 may irreversibly couple the pain-reduction assembly to the needle assembly.

For some applications, pain-reduction assembly 20 is packaged by a manufacturer with a substance disposed in the vicinity of the protruding members (e.g., bristles 22). For example, the substance may be disposed on tips of the bristles, between bristles (e.g., between closely packed bristles), inside bristles that define hollow spaces therein, and/or on an absorbent material (e.g., a sponge) that is disposed between the bristles. For example, an antiseptic agent may be disposed in the vicinity of the bristles. For example, an antiseptic that is bacteriostatic and/or bactericidal may be used, such as, isopropyl alcohol, betadine, and/or chlorhexidine gluconate (CHG). Thus, for some applications, during procedures in which pain-reduction assembly 20 is used, the health-care professional who is carrying out the procedure is not required to administer antiseptic to the needle-insertion site before the pain reduction assembly is placed on the patient's skin. Rather, subsequent to coupling the pain-reduction assembly to the needle assembly, the health-care professional rubs the bristles over the insertion site, thereby antisepticizing the skin-insertion site, and reducing pain associated with the needle insertion, as described hereinabove. Alternatively or additionally, an analgesic and/or an anesthetic may be disposed in the vicinity of the bristles.

Typically, pain-reduction assembly 20 is packaged in a sealed package to maintain the sterility of the pain-reduction assembly, and/or to preserve a substance that is disposed thereon (such as the substances described hereinabove). For example, the sealed package may include a film that is formed over the bristles, and/or a plastic cover that is placed around the pain-reduction assembly.

For some applications, at least one of bristles 22 is an optical fiber, and/or at least one optical fiber is inserted through at least one of the bristles. For example, the optical fiber may be used to provide the healthcare professional with improved visualization of the patient's skin in the vicinity of insertion site 14, by a light-source (e.g., an LED light source) directing light toward the vicinity of the insertion site, via the optical fiber. For some applications, the bristles are configured to facilitate gripping of the patient's skin by the bristles (e.g., by having rough tips), for example, in order for the bristles to be used to tighten the patient's skin in the vicinity of the needle insertion site, as described hereinbelow.

Typically, pain-reduction assembly 20 is shaped to define a space 27 between support arms 24A and 24B. The space between the support arms provides the healthcare professional who is performing the needle insertion with a view of the needle and of the needle insertion point. In addition, the space provides the healthcare professional with access to the needle. In applications in which the pain-reduction assembly is used with intravenous catheter 12, the space provides the healthcare professional with access to a tab 29 of the catheter, which is used to advance the catheter over needle 10.

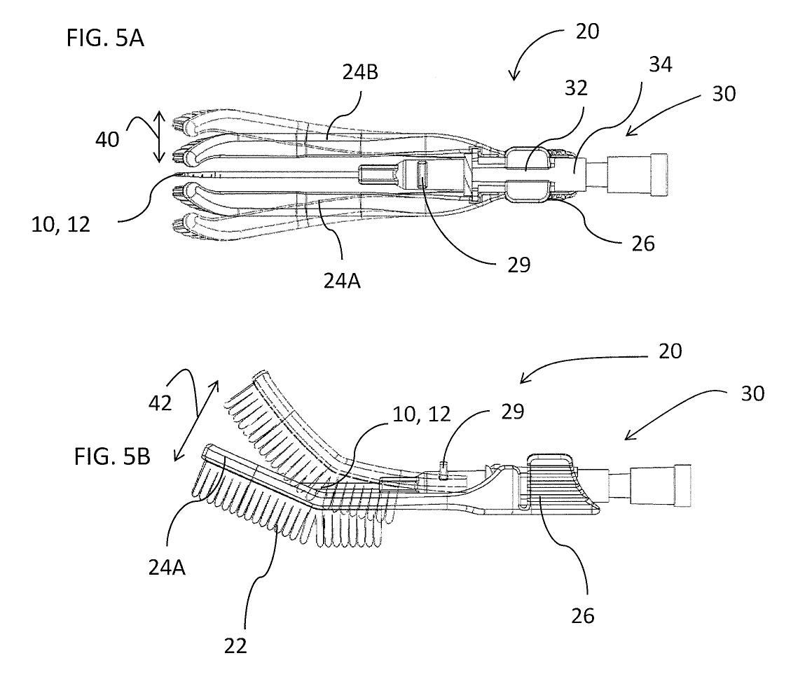

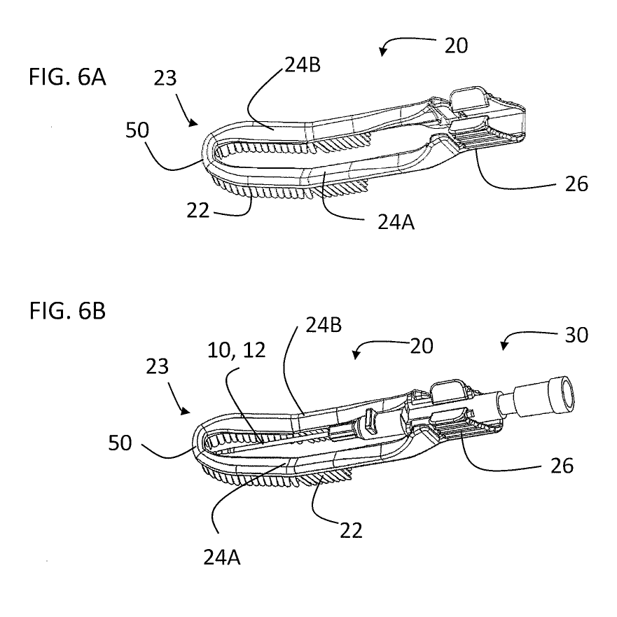

For some applications, as shown, support arms 24A and 24B are not coupled to one another at distal ends thereof. Rather, as shown, pain-reduction assembly is shaped to define an opening 28 in between the distal ends of the support arms. For some applications, the pain-reduction assembly is configured such that intravenous catheter 12 can be slid over needle 10 through the opening between the distal ends of the support arms, as shown in FIG. 1B. Subsequently, needle 10 and pain-reduction assembly 20 are removed from the needle insertion site, and the intravenous catheter is left in the patient's skin. For some applications, support arms 24 are flexible (i.e., the support arms comprise flexible elongate elements), the flexibility of the support arms facilitating the insertion of catheter 12 via opening 28, and/or facilitating tightening of the skin, as described with reference to FIG. 5A. It is noted that for some applications, support element 23 has a generally similar shape to that shown in FIG. 5A, however, the distal end of the support element is closed, such that the support element forms a U-shape, the base of the U being disposed at the distal end of the support element, as shown in FIGS. 6A-B.

Typically, the protruding members (e.g., bristles 22) are movable with respect to the patient's skin, even when the protruding members are engaging the patient's skin. Thus, as described hereinabove, during a typical procedure the protruding members are placed such that they engage the patient's skin, and the protruding members are then advanced over the skin, while engaging the skin. Typically, the protruding members, and/or the support element are flexible, the flexibility of the protruding members and/or the support arms at least partially facilitating the movability of the protruding members with respect to the patient's skin, while the protruding members are engaging the skin.

It is noted that although pain-reduction assembly 20 is shown in the figures as including bristles as the protruding members that protrude from the support element (e.g., support arms 24), the scope of the present invention includes using any protruding members for this purpose, mutatis mutandis. For example, as an alternative or in addition to bristles 22, a coarse material (such as sandpaper) may be disposed on the support element, particles (e.g., sand particles) of the coarse material comprising the protruding members. For some applications, the coarse material is disposed on a material that provides cushioning, such as a foam material. Alternatively, the protruding members may comprise a serrated edge of support element 23 (e.g., a serrated edge of support arms 24A and 24B). Further alternatively, the protruding members may include plastic, metal, and/or another suitable material that has been molded. For example, the material may be molded so as to define spikes. Still further alternatively, the protruding members may comprise Velcro® (e.g., the hook portion of a hook-and-loop fastening member).

It is further noted that the protruding members (e.g., bristles 22) may be any suitable color, size, thickness, stiffness, and/or shape. For some applications, the characteristics of the bristles of a single pain-reduction assembly vary. For example, the stiffness and/or the color of the bristles may vary from the distal end to the proximal end of the assembly. For some applications, the bristles of a given pain-reduction assembly may be color-coded, in order to indicate which needle assemblies are suitable for use with the pain-reduction assembly. Alternatively or additionally, the bristles are colored due to other considerations, e.g., marketing considerations, and/or to give the device a child-friendly appearance. Depending on the use of the pain-reduction assembly (e.g., the area of the patient's body on which the pain-reduction assembly will be used, and/or the needle-assembly with which the pain-reduction assembly will be used), the bristles may vary in texture, for example, the bristles may be stiff, very flexible, of medium flexibility, and/or barbed. Typically, the length of the bristles is sufficient so as to provide flexibility to the bristles.

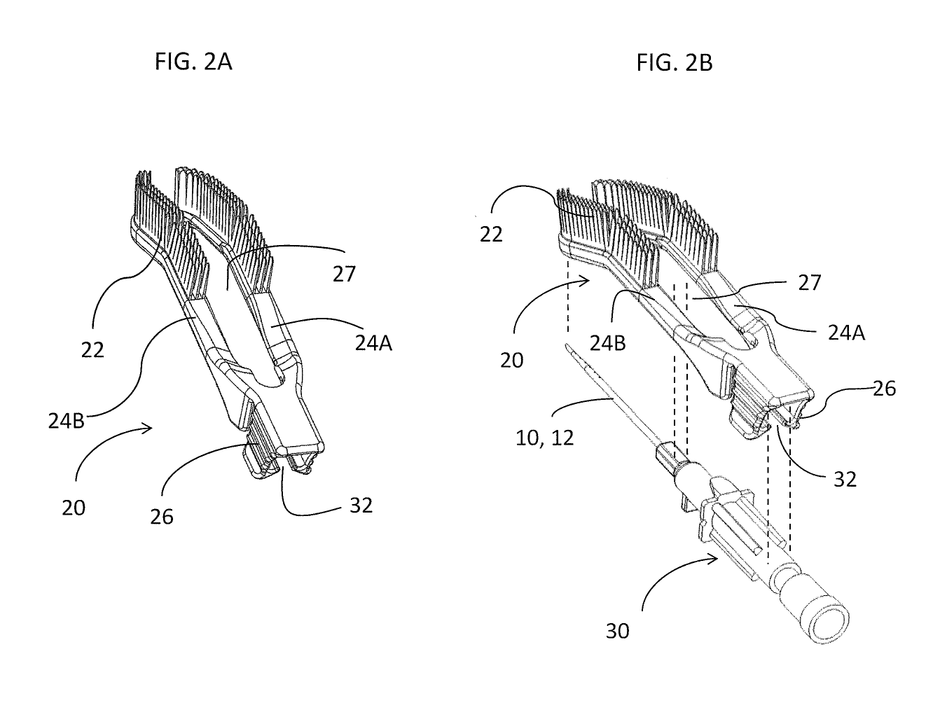

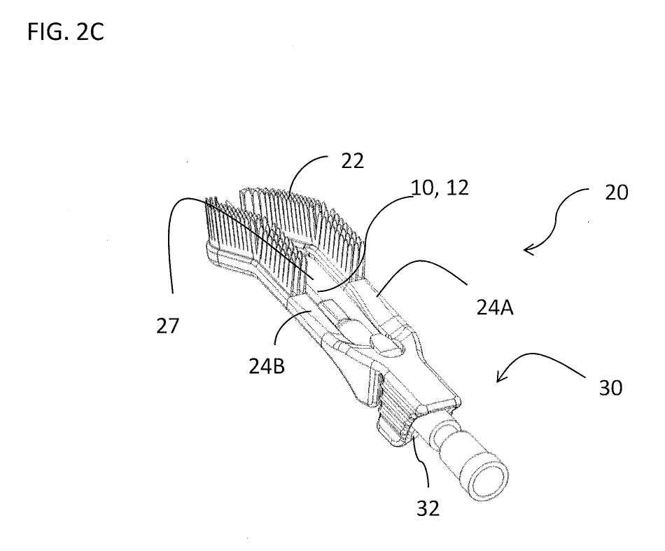

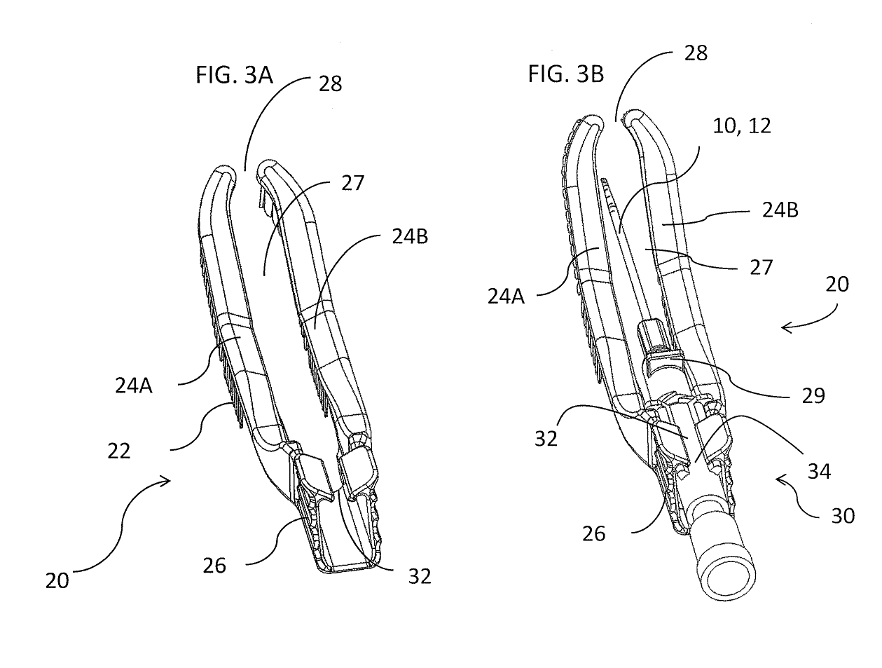

Reference is now made to FIGS. 2A-C, which are schematic illustrations of bottom views of pain-reduction assembly 20, respectively, before needle assembly 30 is coupled to the pain-reduction assembly, during the coupling of the needle assembly to the pain-reduction assembly, and subsequent to the coupling of the needle assembly to the pain-reduction assembly, in accordance with some applications of the present invention. Reference is also made to FIGS. 3A-B, which are schematic illustrations of top views of pain-reduction assembly 20, respectively, before needle assembly 30 is coupled to the pain-reduction assembly, and subsequent to the coupling of the needle assembly to the pain-reduction assembly, in accordance with some applications of the present invention.

For some applications, as shown, pain-reduction assembly 20 is coupled to needle assembly 30 via needle-coupling portion 26 of the pain-reduction assembly, the needle-coupling portion including flexible snap-on arms, which grip the needle assembly. The flexible arms are shaped to define an opening 32 therebetween, the needle assembly being inserted into the opening between the arms. Typically, as shown in FIGS. 2A-3B, the flexible arms are shaped to define opening 32 on the side of the pain-reduction assembly that is generally visible to the healthcare professional during the procedure. For applications in which the pain-reduction assembly is used with a needle of a blood draw device, this may provide the healthcare professional with a view of a blood chamber 34 (shown in FIG. 3B) of the blood draw device. Alternatively or additionally, opening 32 may provide the healthcare professional with a view of other biological or injectable fluids that are transferred via the needle.

For some applications, an alternative mechanism is used for needle-coupling portion 26. For example, a double-sided adhesive strip may be used to couple pain-reduction assembly 20 to needle-assembly 30. For some applications, the use of an adhesive (or a different mechanism) facilitates the coupling of a given pain-reduction assembly to any one of a plurality of types of needle-assemblies, e.g., the needle-assembly types described hereinabove.

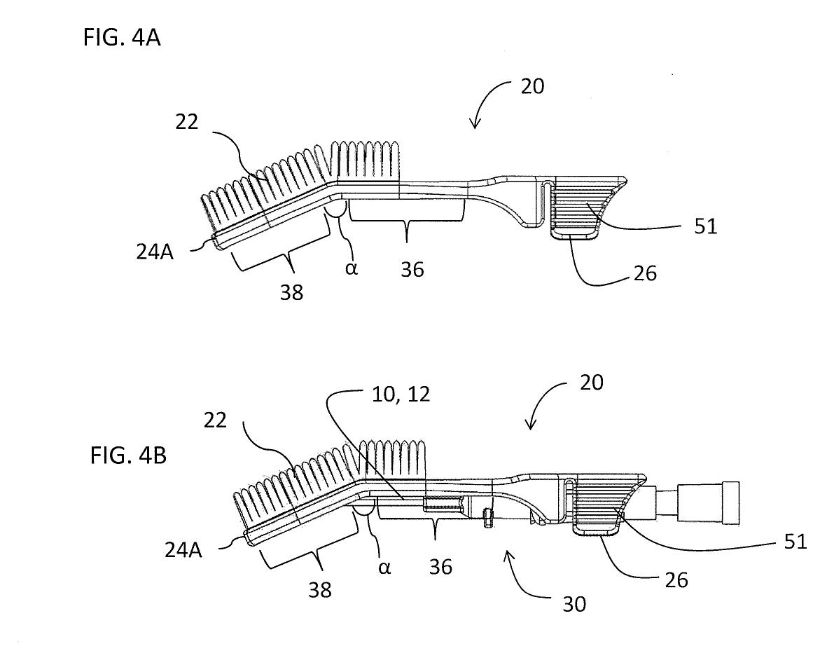

Reference is now made to FIGS. 4A-B, which are schematic illustrations of side views of pain-reduction assembly 20, respectively, before needle assembly 30 is coupled to the pain-reduction assembly, and subsequent to the coupling of the needle assembly to the pain-reduction assembly, in accordance with some applications of the present invention. For some applications, support arms 24A (shown in FIGS. 4A-B) and 24B (not shown in FIGS. 4A-B) comprise proximal portions 36 and distal portions 38 thereof. For some applications, the proximal and distal portions of each of the support arms are disposed at an angle alpha from one another. For some applications, angle alpha is fixed. Alternatively, angle alpha is variable, e.g., for applications in which support arms 24 are flexible (as described hereinbelow with reference to FIG. 5B).

In accordance with respective applications, the support element (e.g., support arms 24A and 24B) defines a plane that is parallel to the needle, or that is disposed at an angle with respect to the needle. For some applications, the disposition of the proximal and distal portions 36 and 38 of support arms 24 is such as to facilitate the insertion of needle 10 into the patient's skin at a given angle or within a given range of angles. For example, the arms may be defined such as to facilitate the insertion of the needle at an angle that facilitates the delivery of drugs or extraction of biological fluids via the needle.

For some applications, the disposition of the proximal and distal portions of the arms at angle alpha from one another facilitates the insertion of the needle at a first angle to the patient's skin, and the subsequent dropping of the needle, to facilitate insertion of catheter 12 into the patient's skin. The bristles on the proximal portions of the support arms typically facilitate pain-reduction during insertion of the catheter into the skin.

For some applications, needle-coupling portion 26 defines bumps and/or indentations that are such as to provide grip 51 for the user of pain-reduction assembly 20.

Reference is now made to FIGS. 5A-B, which are schematic illustrations of pain-reduction assembly 20, support arms 24A and 24B of the pain-reduction assembly being flexible support arms, in accordance with some applications of the present invention.

Typically, support arms 24A and 24B may be flexed laterally (i.e., in directions that are generally parallel with the surface of the skin), as indicated by arrow 40, shown in FIG. 5A. For some applications, the lateral flexing of the support arms facilitates rubbing of the patient's skin by the support arms. Alternatively or additionally, the lateral flexing of the support arms facilitates the insertion of a catheter into the patient's skin by providing the healthcare professional with access to catheter tab 29, as described hereinabove. Further alternatively or additionally, the lateral flexing of the support arms facilitates tightening of the patient's skin in the vicinity of the insertion site of the needle. For example, bristles 22 (or other protruding members) may be configured to grip the patient's skin (e.g., by having small barbs disposed thereon, or by having rough distal surfaces), as described hereinabove. The bristles are placed on the patient's skin on either side of the needle insertion site, and the flexibility of the support arms provides tightening of the skin. Typically, tightening the skin facilitates cannulation of a vein under the skin, since the tightening of the skin reduces the likelihood of the vein rolling underneath the skin, relative to if the skin were not tightened.

Further typically, support arms 24A and 24B may be flexed angularly with respect to needle-coupling portion 26, as indicated by arrow 42, shown in FIG. 5B. For some applications, the angular flexing of the support arms facilitates the insertion of needle 10 into the patient's skin at a desired angle (e.g., an angle that is such as to facilitate delivery or extraction of a substance via the needle, as described hereinabove).

Reference is now made to FIGS. 6A-B, which are schematic illustrations of pain-reduction assembly having a support element 23 with a closed distal end, respectively, before needle assembly 30 is coupled to the pain-reduction assembly, and subsequent to the coupling of the needle assembly to the pain-reduction assembly, in accordance with some applications of the present invention. As shown, the distal end of the support element is closed, such that the support element forms a U-shape, a base 50 of the U being disposed at the distal end of the support element. Support element 23 is generally as described hereinabove, except that support arms 24A and 24B are joined to one another at distal ends thereof. Typically, bristles 22 (or other protruding members) protrude from the bottom surface of the distal end of the support element.

It is noted that, although FIG. 6B shows the support element having the closed distal end being used with an intravenous catheter needle, for some applications, a support element having an opening at its distal end is used with an intravenous catheter needle, to facilitate insertion of the catheter into the subject's skin, as described hereinabove. For some applications, the support element having the closed distal end (shown in FIGS. 6A-B) is used with needles that are placed through the subject's skin, and then removed from the subject's skin a short time later (e.g., within half an hour of the insertion of the needle), leaving nothing inside the subject's skin, for example, blood draw needles.

Experimental Data

An experiment was conducted in which needles were inserted into each of fifteen participants twice: once using a pain-reduction assembly in accordance with the present invention, and once without a pain-reduction assembly. The participants were asked to rate the pain associated with each of the injections using face and verbal pain scales, both of the scales having ranges of one (“no pain”) to 10 (“the worst possible pain”). Four of the participants were blinded, and eleven of the participants were not blinded. Seven of the participants had the needle associated with the pain reduction assembly inserted first, followed by the regular needle, and eight of the participants had the regular needle inserted first.

The mean pain rating using the faces scale was 3.1 when the pain-reduction assemblies were used, versus 3.4 when no pain-reduction assembly was used. The mean pain rating using the verbal scale was 2.8 when the pain-reduction assemblies were used, versus 3.5 when no pain-reduction assembly was used.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with a needle assembly that includes a needle that is for inserting into an insertion site of skin of a patient, the apparatus comprising:
at least one support element;
a needle-coupling portion, coupled to the support element, the needle-coupling portion being configured to couple the support element to the needle assembly; and
a plurality of protruding members that protrude from the support element, the protruding members being configured to engage the skin of the patient, and to be moveable with respect to the skin, while engaging the skin.

2. The apparatus according to claim 1, wherein the protruding members define a serrated edge of the support element.

3. The apparatus according to claim 1, wherein the needle assembly includes a blood-receiving chamber that is configured to receive blood from the patient via the needle, and wherein the needle-coupling portion is configured such that, when the support element is in a coupled state with respect to the needle assembly, a view of the chamber is provided through the needle-coupling portion.

4. The apparatus according to claim 1, wherein the protruding members are flexible and wherein the protruding members are configured to be moveable with respect to the skin while engaging the skin, at least partially due to the flexibility of the protruding members.

5. The apparatus according to claim 1, wherein the support element is at least partially flexible and wherein the protruding members are configured to be moveable with respect to the skin, while engaging the skin, at least partially due to the flexibility of the support element.

6. The apparatus according to claim 1, wherein the protruding members are configured to at least partially obscure a view that the patient has of the needle by at least partially covering the needle.

7. The apparatus according to claim 1, wherein the protruding members are configured to reduce pain associated with the insertion of the needle into the patient's skin, by being moved with respect to the skin.

8. The apparatus according to claim 1, wherein the protruding members are configured to tighten the patient's skin in the vicinity of the insertion site by engaging the skin in the vicinity.

9. The apparatus according to claim 1, wherein the protruding members are configured to reduce a likelihood of a healthcare professional suffering a needlestick injury from the needle, relative to the likelihood of a healthcare professional suffering a needlestick injury from a needle of a needle assembly that is not coupled to the support element, by the protruding members at least partially covering the needle.

10. The apparatus according to claim 1, wherein the support element and the needle assembly form a single integrated unit, by the needle-coupling portion irreversibly coupling the support element to the needle assembly.

11. The apparatus according to claim 1, further comprising a substance disposed in a vicinity of the protruding members, the protruding members being configured to apply the substance to the skin, the substance being selected from the group consisting of an anesthetic, an analgesic, and an antiseptic.

12-14. (canceled)

15. The apparatus according to claim 1, wherein the needle-coupling portion is configured to reversibly couple the support element to the needle assembly.

16. The apparatus according to claim 15, wherein the needle-coupling portion comprises a snap-on mechanism configured to couple the support element to the needle assembly.

17-18. (canceled)

19. The apparatus according to claim 1, wherein the support element is configured such that when the support element is in a coupled state with respect to the needle assembly, a view of the needle is provided through the support element.

20. The apparatus according to claim 19, wherein the support element is shaped to define a space, and wherein the support element is configured such that, when the support element is in a coupled state with respect to the needle, access to the needle is provided via the space.

21. The apparatus according to claim 20, wherein the support element comprises a U-shaped support element that defines the space via which the access to the needle is provided.

22. The apparatus according to claim 20, wherein the support element comprises two arms, the arms being configured to be disposed on respective sides of the needle, when the support element is in a coupled state with respect to the needle.

23. The apparatus according to claim 22, wherein the arms are flexible and are configured to tighten the patient's skin in the vicinity of the insertion site by engaging the patient's skin.

24. The apparatus according to claim 1, wherein the protruding members comprise twenty or more particles of a coarse material that is disposed on the support element.

25. (canceled)

26. The apparatus according to claim 1, wherein the protruding members comprise twenty or more bristles that protrude from the support element.

27. The apparatus according to claim 26, wherein at least one of the bristles comprises a fiber-optic bristle, and wherein the apparatus further comprises at least one light source configured to facilitate visualization of the patient's skin by directing light via the fiber-optic cable.

28. The apparatus according to claim 26, further comprising:
at least one fiber-optic cable configured to be placed inside at least one of the bristles; and
at least one light source configured to facilitate visualization of the patient's skin by directing light via the fiber-optic cable.

29. (canceled)

30. A method for use with a needle assembly that includes a needle that is for inserting into an insertion site of skin of a patient, the method comprising:
placing on the patient's skin, in a vicinity of the insertion site, a plurality of protruding members that protrude from at least one support element, the support element being coupled to the needle assembly; and
subsequently, inserting the needle into the skin, while simultaneously rubbing the skin in the vicinity of the insertion site with the protruding members, by simultaneously advancing distally with respect to the skin in the vicinity, the needle and the support element.

31-56. (canceled)

57. A method for use with a needle assembly that includes a needle that is for inserting into an insertion site of skin of a patient, the method comprising:
placing on the patient's skin, in a vicinity of the insertion site, at least two flexible elongate elements, the elongate elements being coupled to the needle assembly such that the elongate elements are disposed on respective sides of the needle;
stretching the skin in the vicinity by laterally flexing the elongate elements away from one another; and
while the skin is stretched, inserting the needle into the insertion site.
